(19) United States
(12) Patent Application Publication
(10) Pub. No.: US 2026/0183245 A1
(43) Pub. Date: Jul. 2, 2026

(54) CHEWY SOFT CAPSULE

(71) Applicant: ALIMENT INDUSTRY CO., LTD., Minami-koma-gun, Yamanashi (JP)

(72) Inventors: Yohei WAKAO, Minami-koma-gun, Yamanashi (JP); Nanako ONO, Minami-koma-gun, Yamanashi (JP); Akira SUGIMOTO, Minami-koma-gun, Yamanashi (JP); Hiromi MIKAMI, Minami-koma-gun, Yamanashi (JP); Yoshiyuki SHIMOKAWA, Minami-koma-gun, Yamanashi (JP)

(73) Assignee: ALIMENT INDUSTRY CO., LTD., Minami-koma-gun, Yamanashi (JP)

(21) Appl. No.: 19/119,456

(22) PCT Filed: Oct. 10, 2023
(86) PCT No.: PCT/JP2023/036685

§ 371 Date: Mar. 19, 2026

(30) Foreign Application Priority Data
Oct. 11, 2022 (JP) 2022-163224

(51) Int. Cl. A61K 9/48; A61K 47/36

(52) U.S. Cl. CPC A61K 9/4825; A61K 9/4816; A61K 9/4833; A61K 47/36

(57) ABSTRACT

Provided is a chewy soft capsule formulation having pleasant texture, being suppressed from sticking of capsules, adhesion to the container, and discoloring, and having excellent storage stability. The chewy soft capsule formulation includes a capsule shell and a capsule content, wherein the capsule shell contains the following components (A) and (B): (A): from 1 to 35 mass % of gelatin, and (B): from 3 to 25 mass % of an anhydrogalactose-sulfated anhydrogalactose copolymer.

### TECHNICAL FIELD

The present invention relates to a soft capsule formulation that can be chewed in the oral cavity.

### BACKGROUND ART

In recent years, it has been tried to develop soft capsule formulations with the same texture and sense of touch as gummy candy due to expanded use of soft capsules, and soft capsules of which capsule shells can be chewed have been investigated by mixing a larger amount of a plasticizer or mixing starch, a saccharide, or the like.

For example, Patent Literature 1 discloses that a soft capsule preparation having a shell consisting of an oral soft composition containing a gelling agent including gelatin and a polysaccharide thickener, a polyhydric alcohol, a water-soluble polysaccharide, a cellulose, and starch is excellent in texture and sense of touch and is unlikely cause solidification of the composition and adhesion to the container. Patent Literature 2 discloses that a chewy soft capsule formulation prepared by adding a specific plasticizer in an amount several times the amount normally used to gelatin coating and also mixing a water-insoluble cellulose has soft and good chewability and at the same time has low adhesiveness.

However, the solidification of capsules and the adhesion of capsules to the container are still problematic, and problems such that the capsule shell easily discolors are also concerned.

On the other hand, kappa-2 carrageenin is a carrageenan-like polysaccharide thickener which is included in seaweed belonging to Gigartinaceae algae and is known to be a copolymer of anhydrogalactose, which is a part of the structural unit of kappa carrageenan, and sulfated anhydrogalactose, which is a part of the structural unit of iota carrageenan. It has been reported that such kappa-2 carrageenin can be a gelling agent for manufacturing soft capsules as a substitute of gelatin (Patent Literature 3), but there is no report on the use in a capsule shell together with gelatin, it was unclear what type of soft capsules could be formed.

### CITATION LIST

### Patent Literature

Patent Literature 1: JP-A-2021-175740
Patent Literature 2: Japanese Patent No. 3261331
Patent Literature 3: Japanese Patent No. 4558721

### SUMMARY OF INVENTION

### Technical Problem

The present invention relates to providing a chewy soft capsule formulation having pleasant texture, being suppressed from sticking of capsules, adhesion to the container, and discoloring, and having excellent storage stability.

### Solution to Problem

The present inventors have conducted various studies to solve the problem and as a result, have found that a soft capsule formulation formed using a capsule shell including gelatin and an anhydrogalactose-sulfated anhydrogalactose copolymer in specific amounts has texture similar to that of gummy candy when chewed, and the sticking of capsules one another, the adhesion to a container and the discoloring when stored are suppressed.

That is, the present invention relates to the following 1) to 6):
1) A chewy soft capsule formulation comprising a capsule shell and a capsule content, wherein the capsule shell contains the following components (A) and (B):
   (A): from 1 to 35 mass % of gelatin; and
   (B): from 3 to 25 mass % of an anhydrogalactose-sulfated anhydrogalactose copolymer;
2) The chewy soft capsule formulation according to 1), wherein the anhydrogalactose-sulfated anhydrogalactose copolymer (B) is a 3,6-anhydrogalactose-3,6-anhydrogalactose-2-sulfate copolymer;
3) The chewy soft capsule formulation according to 2), wherein the content molar proportion of 3,6-anhydrogalactose-2-sulfate to 3,6-anhydrogalactose (3,6-AG-2-S/3,6-AG) is from 25% to 50%;
4) The chewy soft capsule formulation according to any one of 1) to 3), wherein the content mass ratio of the gelatin (A) and the anhydrogalactose-sulfated anhydrogalactose copolymer (B) [(A):(B)] is from 1:25 to 13:1;
5) The chewy soft capsule formulation according to any one of 1) to 3), wherein the capsule shell contains a plasticizer; and
6) The chewy soft capsule formulation according to any one of 1) to 3), which is a rotary die type soft capsule preparation.

### Advantageous Effects of Invention

According to the present invention, it is possible to provide a chewable soft capsule formulation having pleasant gummy candy-like texture. Further, the above soft capsule formulation is excellent in the storage stability, and the sticking of capsules one another, the adhesion to a container and the discoloring when stored are suppressed.

### DESCRIPTION OF EMBODIMENTS

In the present invention, the gelatin (A) which is a structural component of a capsule shell is a base of the capsule shell and is a modified product of collagen obtained by treating a collagen-derived raw material which is the main protein component of skin, bones, tendons, and so on of cows, sheep, pigs, chickens, fish, and so on, with acid or alkali and then extracting with hot water. As the gelatin used in the present invention, the origin of collagen and the treatment method are not particularly limited.

In the present invention, a hydrolysate or oxygen decomposition product of gelatin or modified gelatin such as succinylated gelatin and phthalated gelatin can also be used, and any type of gelatin may also be suitably used.

The content of the gelatin (A) in the capsule shell is, from the viewpoint of formability and chewiness of the capsule, is 1 mass or more relative to the total amount of the capsule shell composition, preferably 3 mass % or more, more preferably 6 mass % or more and 35 mass % or less, preferably 25 mass % or less, and more preferably 16 mass % or less. In addition, the content is preferably from 1 to 35 mass %, preferably from 3 to 25 mass %, and more preferably from 6 to 16 mass %.

The capsule shell composition is the total amount of the components constituting the shell solution before the capsule formation.

The anhydrogalactose-sulfated anhydrogalactose copolymer (B) used in the present invention is a copolymer of 3,6-anhydrogalactose (3,6-AG) and 3,6-anhydrogalactose-2-sulfate (3,6-AG-2-S) and refers to a copolymer having the content molar proportion of 3,6-anhydrogalactose-2-sulfate to 3,6-anhydrogalactose (3,6-AG-2-S/3,6-AG) of from 25% to 50%.

Such an anhydrogalactose-sulfated anhydrogalactose copolymer is also called kappa-2 carrageenin (R. Falshaw, H. J. Bixler, K. Johndro, Food Hydrocolloids, 15 (2001), 441-452; H. Bizler, K. Johndro, R. Falshaw, Food Hydrocolloids, 15 (2001), 619-630).

The molecular weight of the anhydrogalactose-sulfated anhydrogalactose copolymer is usually 100,000 dalton or more, preferably from 100,000 to 1,000, 000 dalton, more preferably from 100,000 to 450,000 dalton, and further preferably from 100,000 to 350,000 dalton.

The anhydrogalactose-sulfated anhydrogalactose copolymer is included in seaweed belonging to, for example, Gigartinaceae algae (such as Gigartina radula, Gigartina corymbifera, Gigartina skottsbergii, Iridaea cordata, Sarcothalia crispata, and Mazzaella laminarioides), and can be obtained by separating, collecting, and purifying therefrom. The ratio of 3,6-AG can also be appropriately increased by converting the 3,6-AG-2-S portion into 3,6-AG through desulfurization and oxidation by alkali treatment during separation and collection (modification treatment).

The method of separation and collection and the method of modification treatment are well known and are described in the above-mentioned literatures written by Falshaw, Bixler, and Johndro. For example, the modification treatment may be performed by alkali treatment under warming during collection from Gigartinaceae algae.

The anhydrogalactose-sulfated anhydrogalactose copolymer is commercially available as “SeaGel CAP101” (manufactured by FMC Corporation).

The content of the anhydrogalactose-sulfated anhydrogalactose copolymer (B) in the capsule shell is, from the viewpoint of formability and chewiness of the capsule, 3 mass % or more relative to the total amount of the capsule shell composition, preferably 6 mass % or more and more preferably 8 mass % or more and 25 mass % or less, preferably 20 mass % or less, and more preferably 16 mass % or less. Further, the content is preferably from 3 to 25 masse, preferably from 6 to 20 mass %, and more preferably from 8 to 16 mass %.

The content mass ratio of the gelatin (A) and the anhydrogalactose-sulfated anhydrogalactose copolymer (B) [(A):(B)] is preferably from 1:25 to 13:1, more preferably from 1:12 to 6:1, and further preferably from 3:5 to 5:3, from the viewpoint of chewiness and storage stability.

The capsule shell of the present invention preferably contains a plasticizer in order to improve the texture.

Examples of the plasticizer include glycerins (such as glycerin, diglycerin, and polyglycerin), sugar alcohols (such as sorbitol, xylitol, erythritol, maltitol, mannitol, and cyclitol), glycols (such as propylene glycol, dipropylene glycol, 1,3-butylene glycol, ethylene glycol, and polyethylene glycol), disaccharides, and oligosaccharides.

The content of the plasticizer in the capsule shell is preferably 10 mass % or more in the total amount of the capsule shell composition, more preferably 20 mass % or more, and further preferably 30 mass % or more and preferably 60 mass % or less, more preferably 50 mass % or less, and further preferably 45 mass % or less.

The capsule shell can further contain various additives such as a water-soluble polymer, starch, pigments (a natural pigment and a synthetic pigment), various sweeteners, a preservative, a water activity reducing agent, and a pH adjuster, as a case requires.

Examples of the water-soluble polymer include, but not limited to, water-soluble plant fibers derived from plants or seaweed, such as pectin, glucomannan, sodium alginate, pullulan, fucoidan, maltodextrin, isomaltodextrin, gellan gum, locust bean gum, and xanthan gum.

Examples of the starch include unmodified starch, modified starch, and a starch decomposition product. Examples of the origin plant include wheat, rice, barley, glutinous rice, glutinous barley, buckwheat, soybean, potato, tapioca, and corn.

The contents of the water-soluble polymer and starch in the capsule shell are each preferably 6 mass % or less in the total amount of solid contents.

In the present invention, the capsule shell contains a gelatin (A) and an anhydrogalactose-sulfated anhydrogalactose copolymer (B) which have a function as a gelling agent, however, other gelling agents may be contained so long as the chewiness and the storage stability of the capsule are not impaired. Here, examples of other gelling agents include carrageenan, locust bean gum, agar, psyllium seed gum, konjac powder, gellan gum, and xanthan gum.

The chewy soft capsule formulation of the present invention may be a rotary die type soft capsule preparation prepared by forming a shell sheet by drawing a capsule shell composition on a left and right pair of rotating drums using a rotary die type soft capsule forming machine, then applying a temperature appropriate to the shell sheet surface by segments, forming the shell sheet into a predetermined shape using a rotating die roll, and at the same time filling with the content while sticking the shell sheet; a flat plate type soft capsule preparation formed by placing a shell sheet on one of a pair of flat plate dies, placing a content thereon, further covering it with a shell sheet, and finally placing the other die thereon and then pressing into a predetermined shape; or a seamless soft capsule preparation formed by dropping a capsule shell composition and a content simultaneously discharged from dual nozzles present on the same circle into a cooling liquid flowing at a constant rate, but the rotary die type soft capsule preparation is preferable from the viewpoint of a wide range of manufacturable content liquids, a high degree of freedom in choosing shapes and sizes, and the productivity.

The rotary die type soft capsule preparation capsulated using rotary dies can be preferably formed, for example, by injecting a liquid material such as an oily liquid, an aqueous liquid, or a dispersion of powder such as oil-resistant powder in an oily liquid, between sheet-like formed capsulate shell sheets and subjecting it to compressive folding from both sides.

The capsule shell composition may be prepared by stirring and dispersing the gelatin (A), the anhydrogalactose-sulfated anhydrogalactose copolymer (B), and further various additives as a case requires in water, stirring and dissolving them at from 70° C. to 98° C., and then performing vacuum defoaming.

In the chewy soft capsule formulation of the present invention, the content of the capsule is not particularly limited, and examples thereof include materials which are usually filled in capsules such as medicine, quasi-drugs, cosmetics, various ingredients used as foods (health foods, special health foods, foods with functional claims, and supplements), seasonings, and flavoring. The form of the content may be any of solution, suspension, paste, powder, and granules and is preferably solution, suspension, or paste. Examples of the content that can be mixed in the capsule content are shown below.

Examples of the functional oil and fat include ω3 polyunsaturated fatty acids such as DHA, EPA, and α-linolenic acid, ω6 polyunsaturated fatty acids such as linoleic acid and γ-linolenic acid, and oleic acid as a ω9 monovalent unsaturated fatty acid.

Examples of other oil and fat include vegetable oil and fat such as soybean oil, rapeseed oil, safflower oil, rice oil, corn oil, sunflower oil, cottonseed oil, olive oil, sesame oil, peanut oil, adlay oil, wheat germ oil, perilla oil, linseed oil, perilla seed oil, sacha inchi oil, walnut oil, kiwi seed oil, salvia seed oil, parsley seed oil, grape seed oil, macadamia nut oil, hazelnut oil, almond oil, pumpkin seed oil, camellia oil, tea seed oil, borage oil, palm oil, palm olein, palm stearin, coconut oil, palm kernel oil, cocoa butter, sal butter, shea butter, and algae oil; animal oil and fat such as fish oil, lard, beef tallow, and butter fat; and transesterified, hydrogenated, and fractionated oils thereof.

In addition, examples of the material that is used in supplements include ginkgo leaf extract, phosphatidylserine, GABA, chicken-derived plasmalogen, anthocyanin-containing bilberry or blackcurrant extract, lutein and zeaxanthin-containing marigold pigment, astaxanthin-containing Haematococcus algae pigment, crocetin-containing Gardenia pigment, carotenoids such as α-carotene, β-carotene, lycopene, and β-cryptoxan, quinones such as vitamin K, coenzyme Q10, and PQQ, polyphenols such as flavonol, isoflavone, tannin, catechin, quercetin, anthocyanin, flavangenol, and flavonoid, cannabidiol as a cannabinoid, propolis, xylitol, vitamins such as vitamins B1 and B2, niacin, pantothenic acid, vitamin B6, vitamin B12, vitamin C, vitamin D3, and vitamin E, minerals such as calcium, phosphorus, sodium, potassium, magnesium, zinc, selenium, chromium, molybdenum, iron, and copper, lactic acid bacteria such as L-92 Lactobacillus, Plasma Lactobacillus, Lactobacillus casei Shirota strain, Lactobacillus gasseri SP strain, and LG-21 Lactobacillus, and Lactobacillus bifidus such as BB-536 strain and B-3 strain.

Examples of the medicine include meclizine hydrochloride, scopolamine hydrobromide hydrate, scopolia extract, berberine tannate, and sodium fluoride.

The thus-obtained chewy soft capsule formulation has excellent storage stability and is packed in various packaging formats such as bottled packaging, PTP packaging, and pouching and is stored and distributed.

### EXAMPLES

The present invention will now be described specifically with reference to Examples, but the present invention is not limited thereto in any way. Examples 1 to 6 and Comparative Examples 1 to 9

### 1. Raw Material

1) Anhydrogalactose-sulfated anhydrogalactose copolymer: SeaGel CAP101 (manufactured by FMC Corporation)
2) Gelatin (derived from pig): BCN200S (manufactured by Nitta Gelatin Inc.)
3) Gelatin (derived from cow): NSC150 (manufactured by Nitta Gelatin Inc.)
4) Gellan gum: KELCOGEL CG-LA (manufactured Sansho Co., Ltd.)
5) Gellan gum (native): KELCOGEL CG-HA (manufactured Sansho Co., Ltd.)
6) Plasticizer: glycerin (manufactured by Sakamoto Yakuhin Kogyo Co., Ltd.)
7) Carrageenan (mixture of κ and ι): GENUTINE 310-C (manufactured Sansho Co., Ltd.)
8) Carrageenan (κ): GENUGEL carrageenan type WR-78-J (manufactured Sansho Co., Ltd.)
9) Carrageenan (ι): GENUGEL carrageenan type CJ (manufactured Sansho Co., Ltd.)
10) Carrageenan (λ): SATIAGEL (trademark) BDC 20 (manufactured by Unitec Foods Co., Ltd.)
11) Sodium carboxymethylcellulose: SUNROSE SLD-FM (manufactured by Nippon Paper Industries Co., Ltd.)
12) Starch: Rice starch (manufactured by Japan Corn Starch Co., Ltd.)
13) Konjac powder: Fine super mannan (manufactured by Ogino Shoten K.K.)
14) Isomaltodextrin: Fibryxa (registered trademark) (manufactured by Hayashibara Co., Ltd.)
15) Sugar: Frost Sugar FS-2 (manufactured by Nissin Sugar Manufacturing Co., Ltd.)
16) Polyethylene glycol: Polyethylene Glycol 400/4000 (manufactured by FUJIFILM Wako Pure Chemical Corporation)
17) Crystalline cellulose: Ceolus (manufactured by Asahi Kasei Corporation)

### 2. Production of Shell Solution

### 2-1. Examples 1 to 6 and Comparative Examples 3 to 5

Glycerin was weighed in a prescribed container, and an anhydrogalactose-sulfated anhydrogalactose copolymer was added thereto little by little while stirring and was uniformly dispersed to obtain an anhydrogalactose-sulfated anhydrogalactose copolymer dispersion. Purified water and the anhydrogalactose-sulfated anhydrogalactose copolymer dispersion were put in a mixing tank and dissolved while stirring. Subsequently, gelatin (and gellan gum in Examples 5 and 6) was added thereto and dissolved therein while stirring, and deaeration was performed by agitation defoaming to obtain a capsule shell composition.

### 2-2. Comparative Example 1

A part of glycerin was added in a prescribed container, and konjac powder, sodium carboxymethylcellulose, and rice starch were added thereto in this order little by little while stirring, and stirring was continued until uniformly dispersed to obtain a dispersion. Purified water, the remaining glycerin, and isomaltodextrin were weighed and added in a mixing tank and dissolved while stirring. Subsequently, gelatin was added thereto and completely dissolved while stirring. The dispersion was added thereto and dissolved therein little by little while continuously stirring, and deaeration was performed by agitation defoaming to obtain a capsule shell composition.

### 2-3. Comparative Example 2

Purified water, glycerin, and sugar were weighed in a prescribed container and stirred to completely dissolve. Subsequently, polyethylene glycol was added thereto and stirred and dissolved, and crystalline cellulose was further added thereto and dispersed therein. Subsequently, gelatin was added thereto while stirring and uniformly dissolved to obtain a capsule shell composition.

### 2-4. Comparative Examples 6 to 9

Glycerin was weighed in a prescribed container, and carrageenan was added thereto little by little while stirring and uniformly dispersed to obtain a carrageenan dispersion. Purified water and the carrageenan dispersion were put in a mixing tank and dissolved while stirring. Subsequently, gelatin was added thereto and dissolved while stirring, and deaeration was performed by agitation defoaming to obtain a capsule shell composition.

### 3. Capsule Forming

The capsule shell compositions prepared in the above 2 were each filled in the spreader box of a rotary die type soft capsule forming machine and drawn on the rotating drum into a thin film of about 0.8 mm, followed by cooling to obtain respective shell sheets.

Subsequently, MCT oil (medium chain triglyceride oil) as the content was filled in immediately before punching while warming and sticking the shell sheet surface by segments, and the formed product by punching was dried to produce a soft capsule formulation.

### 4. Drying

The formed soft capsule formulation was sent to a tumbler dryer connected to a soft capsule forming machine and was dried at a temperature of from 20° C. to 30° C. and a relative humidity of from 5% to 50% for from 8 to 60 hours to obtain an objective soft capsule formulation.

### 5. Evaluation Test

### (1) Formability of Capsule

The formability of a capsule was judged by whether a soft capsule could be formed using a rotary die type soft capsule forming machine or not:
◯: a soft capsule could be formed; and
x: a soft capsule could not be formed.

### (2) Chewiness

Chewiness was evaluated by four expert panelists on three criteria: “soft and chewable”, “chewable, but slightly hard”, and “hard and unchewable”:

◯: soft and chewable;
Δ: chewable, but slightly hard; and
x: hard and unchewable.

### (3) Storage Stability

Sixty capsules of each of the produced soft capsule formulations were placed in respective glass bottles, and the bottles were capped and stored in a thermohygrostat of a room temperature of 40° C. and a humidity of 75% for 4 months, and the appearance of the soft capsule formulation and adhesion of the capsules and to the container wall were evaluated.

### 1) Appearance

In evaluation of appearance, each sample was taken out from a storage and was left to stand at room temperature for 24 hours, and whether a color change (browning) occurred or not was visually verified. Four expert panelists performed the evaluation on 3 criteria: “no change”, “slight change”, and “significant change”:
◯: no change;
Δ: slight change; and
x: significant change.

### 2) Adhesiveness

In evaluation of adhesiveness, each sample was taken out from the storage and was left to stand at room temperature for 24 hours, the cap was then opened and the content, i.e., the capsules, in the bottle was spread on an aluminum tray. Then, capsules were judged as “no adhesion” when all of the capsules could be taken out only by lightly shaking the bottle, capsules were judged as “slight adhesion” when a small amount of the capsules adhered and remained in the bottle only by lightly shaking the bottle, and capsules were judged as “significant adhesion” when the capsules could not be taken out only by lightly shaking the bottle:
◯: no adhesion;
Δ: slight adhesion; and
x: significant adhesion.

TABLE 1-1

| | | | Example | Example | Example | Example | Example | Example | Comparative | Comparative |
|---|---|---|---|---|---|---|---|---|---|---|
| | | | 1 | 2 | 3 | 4 | 5 | 6 | Example 1 | Example 2 |
| Prescription | Anhydrogalactose-sulfated | | 11 | 11 | 11 | 11 | 10 | 10 | — | — |
| (%) | anhydrogalactose copolymer | | | | | | | | | |
| | Gelatin (derived from pig) | | 11 | 16 | 2 | — | 10 | 10 | 31 | 22 |
| | Gelatin (derived from cow) | | — | — | — | 11 | — | — | — | — |
| | Gellan gum | | — | — | — | — | 2 | — | — | — |
| | Gellan gum (native) | | — | — | — | — | — | 2 | — | — |
| | Glycerin | | 36 | 25 | 40 | 36 | 36 | 36 | 31 | 18 |
| | Carrageenan (κ, ι mixture) | | — | — | — | — | — | — | — | — |
| | Carrageenan (κ) | | — | — | — | — | — | — | — | — |
| | Carrageenan (ι) | | — | — | — | — | — | — | — | — |
| | Carrageenan (λ) | | — | — | — | — | — | — | — | — |
| | Sodium carboxymethylcellulose | | — | — | — | — | — | — | 2 | — |
| | Starch | | — | — | — | — | — | — | 2 | — |
| | Konjac powder | | — | — | — | — | — | — | 1 | — |
| | Isomaltodextrin | | — | — | — | — | — | — | 3 | — |
| | Sugar | | — | — | — | — | — | — | — | 9 |
| | Polyethylene glycol | | — | — | — | — | — | — | — | 18 |
| | Crystalline cellulose | | — | — | — | — | — | — | — | 11 |
| | Purified water | | 42 | 48 | 47 | 42 | 42 | 42 | 30 | 22 |
| Evaluation | Formability of capsule | | ∘ | ∘ | ∘ | ∘ | ∘ | ∘ | ∘ | ∘ |
| result | Chewiness | | ∘ | ∘ | ∘ | ∘ | ∘ | ∘ | ∘ | ∘ |
| | Storage stability | Appearance | ∘ | ∘ | ∘ | ∘ | ∘ | ∘ | x | x |
| | (40° C./4M) | Adhesiveness | ∘ | ∘ | ∘ | ∘ | ∘ | ∘ | x | x |

TABLE 1-2

| | | | Comparative | Comparative | Comparative | Comparative | Comparative | Comparative | Comparative |
|---|---|---|---|---|---|---|---|---|---|
| | | | Example 3 | Example 4 | Example 5 | Example 6 | Example 7 | Example 8 | Example 9 |
| Prescription | Anhydrogalactose-sulfated | | 3 | 30 | 2 | — | — | — | — |
| (%) | anhydrogalactose copolymer | | | | | | | | |
| | Gelatin | | 39 | 30 | 5 | 11 | 11 | 11 | 11 |
| | Glycerin | | 30 | 20 | 40 | 36 | 36 | 36 | 36 |
| | Gelatin (derived from cow) | | — | — | — | — | — | — | — |
| | Gellan gum | | — | — | — | — | — | — | — |
| | Gellan gum (native) | | — | — | — | — | — | — | — |
| | Carrageenan (κ, ι mixture) | | — | — | — | 11 | — | — | — |
| | Carrageenan (κ) | | — | — | — | — | 11 | — | — |
| | Carrageenan (ι) | | — | — | — | — | — | 11 | — |
| | Carrageenan (λ) | | — | — | — | — | — | — | 11 |
| | Sodium carboxymethylcellulose | | — | — | — | — | — | — | — |
| | Starch | | — | — | — | — | — | — | — |
| | Konjac powder | | — | — | — | — | — | — | — |
| | Isomaltodextrin | | — | — | — | — | — | — | — |
| | Sugar | | — | — | — | — | — | — | — |
| | Polyethylene glycol | | — | — | — | — | — | — | — |
| | Crystalline cellulose | | — | — | — | — | — | — | — |
| | Purified water | | 28 | 20 | 53 | 42 | 42 | 42 | 42 |
| Evaluation | Formability of capsule | | ∘ | x | x | x | x | x | x |
| result | Chewiness | | x | Unevaluable | Unevaluable | Unevaluable | Unevaluable | Unevaluable | Unevaluable |
| | Storage stability | Appearance | ∘ | Unevaluable | Unevaluable | Unevaluable | Unevaluable | Unevaluable | Unevaluable |
| | (40° C./4M) | Adhesiveness | ∘ | Unevaluable | Unevaluable | Unevaluable | Unevaluable | Unevaluable | Unevaluable |

As shown in Tables 1-1 and 1-2, the soft capsule formulations of the present invention in Examples 1 to 6 were excellent in capsule formability and had soft texture and good chewiness when chewed. The appearance was not significantly changed even after storing at 40° C. for 4 months, no adhesion between capsules nor adhesion of capsules to the container was observed, and the storage stability was excellent.

On the other hand, in Comparative Example 1 in which the shell contained starch, although the formability and chewiness of the capsule were good, the shell was significantly browned after storing at 40° C. for 4 mounts, and solidification of the capsules and adhesion of the capsules to the container were observed. Thus, it was demonstrated that there is a problem in the storage stability.

In Comparative Example 2 in which the shell contained sugar or the like, as in Comparative Example 1, although the formability and chewiness of the capsule were good, there was a problem in the storage stability.

In Comparative Example 3 in which an anhydrogalactose-sulfated anhydrogalactose copolymer and gelatin were added in amounts of 3% and 39%, respectively, although the formability of the capsule was good, the capsule was hard and could not be easily chewed. Thus, there was a problem in the chewiness.

In Comparative Example 4 in which an anhydrogalactose-sulfated anhydrogalactose copolymer and gelatin were added in amounts of 30% and 30%, respectively, the solution solidified during producing the capsule shell composition. Thus, there was a problem in the formability of the capsule.

In Comparative Example 5 in which an anhydrogalactose-sulfated anhydrogalactose copolymer and gelatin were added in amounts of 2% and 5%, respectively, although a capsule shell composition could be produced, the strength of the shell sheet was low, and the adhesion between the shells when forming the capsule was poor. Thus, there was a problem in the formability of the capsule.

In Comparative Examples 6, 7, 8, and 9 in which the shells contained generally known carrageenan, the solution solidified when producing the capsule shell composition. Thus, there was a problem in the formability of the capsule.

1. A chewy soft capsule formulation comprising a capsule shell and a capsule content, wherein the capsule shell contains the following components (A) and (B):
(A): from 1 to 35 mass % of gelatin; and
(B): from 3 to 25 mass % of an anhydrogalactose-sulfated anhydrogalactose copolymer.

2. The chewy soft capsule formulation according to claim 1, wherein the anhydrogalactose-sulfated anhydrogalactose copolymer (B) is a 3,6-anhydrogalactose-3,6-anhydrogalactose-2-sulfate copolymer.

3. The chewy soft capsule formulation according to claim 2, wherein 3,6-AG-2-S/3,6-AG, which is a content molar proportion of 3,6-anhydrogalactose-2-sulfate to 3,6-anhydrogalactose in the 3,6-anhydrogalactose-3,6-anhydrogalactose-2-sulfate copolymer, is from 25% to 50%.

4. The chewy soft capsule formulation according to claim 1, wherein [(A):(B)], which is a content mass ratio of the gelatin (A) and the anhydrogalactose-sulfated anhydrogalactose copolymer (B), is from 1:25 to 13:1.

5. The chewy soft capsule formulation according to claim 1, wherein the capsule shell contains a plasticizer.

6. The chewy soft capsule formulation according to claim 1, which is a rotary die type soft capsule preparation.
